(19) United States
(12) Patent Application Publication
(10) Pub. No.: US 2025/0186043 A1
(43) Pub. Date: Jun. 12, 2025

(54) LAYERED ADJUNCTS

(71) Applicant: CILAG GMBH INTERNTIONAL, ZUG (CH)

(72) Inventors: JOHN K. BRUCE, MORROW, OH (US); NICHOLAS J. ROSS, FRANKLIN, OH (US); JONATHAN KOVAL, CINCINNATI, OH (US); ZHIFAN F. HUANG, MASON, OH (US); THOMAS A. STOUGHTON, CINCINNATI, OH (US); ATUL M. GODBOLE, LIBERTY TOWNSHIP, OH (US)

(21) Appl. No.: 18/530,885

(22) Filed: Dec. 6, 2023

(51) Int. Cl. A61B 17/072; A61B 17/00

(52) U.S. Cl. CPC A61B 17/072; A61B 2017/00889; A61B 2017/00893; A61B 2017/07271; A61B 2017/07278; A61B 2017/07285

(57) ABSTRACT

The disclosed technology includes a staple cartridge having an elongate body and an implantable pouch. The elongate body includes a central slit, a deck, a plurality of staple pockets, and a plurality of staples. The driver includes at least one of a sled or a knife and can be configured to translate through at least a portion of the central slit. The plurality of staples are disposed within the plurality of staple pockets. The implantable pouch includes a medicant disposed therein. The implantable pouch is removably secured to the elongate body and is positioned to be ruptured by at least one of the driver when it translates through at least the portion of the central slit or at least one of the plurality of staples ejected from one of the plurality of staple pockets thereby releasing the medicant.

### FIELD

The present invention relates generally to layered adjuncts for surgical instruments and more specifically an implantable pouch affixed to a surgical instrument to administer deliverable agents to a patient's tissue.

### BACKGROUND

Surgical instruments, specifically surgical staplers, face several challenges when utilized by practitioners during procedures. When employed, surgical staplers perform the functions of clamping a patient's tissue at a desired point, cutting the patient's tissue at the desired point, and then stapling the patient's tissue at the desired point. The purpose of clamping the tissue prior to use is to create an even topography of tissue, which promotes effective cutting and stapling of the patient's tissue. However, during clamping, the surgical stapler may encounter tissue of varying thickness near the incision. Consequently, the surgical stapler, unable to compensate for the varying thickness of the tissue, may create uneven excision and stapling of the tissue that can result in undesirable leakage or bleeding. In the event that leakage or bleeding is observed during surgery, for example, the practitioner may elect to extend the length of a procedure on a patient, resulting in longer post-operation (post-op) healing times for the patient.

Another challenge that may exist during procedures is the administration of deliverable agents to patients in vivo. Hemostasis agents, chemotherapy agents, anti-inflammatory agents, and the like may be administered to incisions, and many of these agents require mixing prior to delivery to the patient. Current surgical stapler technologies lack a method or device capable of mixing and/or administering deliverable agents to patients in vivo concurrently with the cutting and stapling procedure. Resultantly, the inability to administer deliverable agents with surgical staplers may create longer post-op healing times, make targeted pain management arduous, and increase risk of infection. These and other problems exist.

### SUMMARY

The technology disclosed herein addresses the aforementioned challenges. There is provided, in accordance with an example of the present technology, a staple cartridge that can comprise an elongate body and an implantable pouch. The elongate body can comprise a central slit, a deck, a driver, a plurality of staple pockets, and a plurality of staples. The driver can comprise at least one of a sled or a knife and can be configured to translate through at least a portion of the central slit. Each of the staple pockets are accessible via an opening defined by the deck. The plurality of staples can be disposed within the plurality of staple pockets. The implantable pouch can comprise a medicant disposed therein. The implantable pouch can be removably secured to the elongate body and positioned to be ruptured by at least one of the driver when it translates through at least the portion of the central slit or at least one of the plurality of staples as the at least one of the plurality of staples is ejected from one of the plurality of staple pockets. Rupturing of the implantable pouch by either the driver or at least one of the plurality of staples can release the medicant.

There is provided, in accordance with an example of the present technology a method for applying a medicant onto an end effector of a surgical instrument for delivery to a targeted tissue, the method can comprise: assembling the end effector. The end effector can comprise a channel jaw; an anvil jaw, and a staple cartridge disposed in the channel jaw. The anvil jaw can be hingedly connected to the channel jaw and can be moveable between an open position and a closed position with respect to the channel jaw. The staple cartridge can comprise an elongate body. The elongate body can comprise a central slit, a driver, a deck, a plurality of staple pockets, a plurality of staples, and an implantable pouch.

The driver can comprise at least one of a sled or a knife. The driver can be configured to translate through at least a portion of the central slit. Each of the plurality of staple pockets can be accessible via an opening defined by the deck. The plurality of staples can be disposed within the plurality of staple pockets. The implantable pouch can comprise the medicant. The method can further comprise adhering the implantable pouch comprising the medicant to the staple cartridge. The method can further comprise clamping the targeted tissue with the anvil jaw while in the closed position and the implantable pouch being disposed therebetween the anvil jaw and the targeted tissue. The method can further comprise rupturing the implantable pouch comprising the medicant with the plurality of staples while the anvil jaw is in the closed position. The method can further comprise delivering the medicant via the plurality of staples to the targeted tissue.

Additional features, functionalities, and applications of the disclosed technology are discussed in more detail herein.

### BRIEF DESCRIPTION OF THE DRAWINGS

FIG. 1 is a schematic pictorial illustration of a surgical instrument, end effector, and a staple cartridge, in accordance with an example of the disclosed technology;

FIG. 2 is a top view illustration of features of a staple cartridge, in accordance with an example of the disclosed technology;

FIG. 3 is a perspective view of a staple cartridge and an implantable pouch installed within the end effector of a surgical instrument while the end effector is in an open position, in accordance with an example of the disclosed technology;

FIG. 4 is a perspective, cross-sectional view of a multi-ply implantable pouch containing a medicant separated into one or more layers prior to being mixed, in accordance with an example of the disclosed technology;

FIG. 5 is a perspective view of a staple cartridge installed within the end effector of a surgical instrument while in the closed position with a targeted tissue disposed therebetween, in accordance with an example of the disclosed technology;

FIG. 6 is a perspective, cross-sectional view of a multi-ply implantable pouch containing medicant in one or more layers after being mixed, in accordance with an example of the disclosed technology;

FIG. 7A is a schematic of a multi-ply implantable pouch containing medicant in one or more layers in accordance with an example of the disclosed technology;

FIG. 7B is a schematic of the multi-ply implantable pouch of FIG. 7A being ruptured by a staple thereby causing the implantable pouch to swell, in accordance with an example of the disclosed technology;

FIG. 8 is an illustration of a staple within a staple pockets rupturing an implantable pouch containing medicant and carrying the medicant to the targeted tissue, in accordance with an example of the disclosed technology;

FIG. 9 is a side, cross-sectional illustration of a sled and a blade of the driver configured to traverse central slit of the staple cartridge and rupture an implantable pouch containing a medicant, in accordance with an example of the disclosed technology;

FIG. 10 is a flow chart showing a method for applying a medicant onto an end effector of a surgical instrument for delivery to a targeted tissue; in accordance with an example of the disclosed technology.

### DETAILED DESCRIPTION

The following detailed description should be read with reference to the drawings, in which like elements in different drawings are identically numbered. The drawings, which are not necessarily to scale, depict selected examples and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives, and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 71% to 110%. In addition, as used herein, the terms “patient,” “host,” “user,” and “subject” refer to any human or animal subject and are not intended to limit the systems or methods to human use, although use of the subject invention in a human patient represents a preferred embodiment. As well, the term “proximal” indicates a location closer to the operator or physician (e.g., closer to a handle held by the operator) whereas “distal” indicates a location further away to the operator or physician. As discussed herein, “physician” can include a doctor, surgeon, technician, scientist, operator, or any other individual associated with using or operating a surgical stapler.

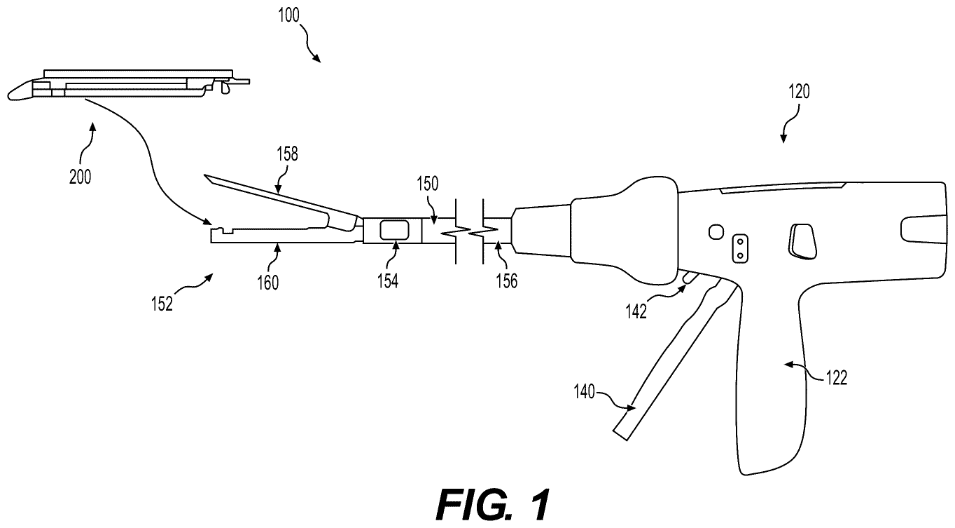

FIG. 1 is a schematic pictorial illustration of a surgical instrument 100, end effector 152, and a staple cartridge 200. For exemplary purposes, the surgical instrument 100 of the present disclosure may be understood to be a surgical stapler that can both cut and staple along a path of tissue; however, it should be appreciated that the present disclosure is not so limited. As one skilled in the art will appreciate, the devices described herein can include devices configured to perform other endoscopic procedures on a patient, which may include one of cutting or stapling. As illustrated in FIG. 1, the surgical instrument 100 may include a housing 120, a handle portion 122, a first trigger 140, a second trigger 142, and a shaft 150. The shaft 150 may include a distal end 154, which can communicably attach with an end effector 152 of the surgical instrument 100, and a proximal end 156, which can communicably attach with the housing 120.

The end effector 152 may include an anvil jaw 158 and a channel jaw 160. The anvil jaw 158 may be hingedly connected to the channel jaw and can be configured to be movable between and open and a closed position with respect to the channel jaw 160. The first trigger 140, upon transmitting an activation signal to the end effector 152, can be configured to transition the anvil jaw 158 between the open position and closed position. For example, as illustrated in FIG. 1, the anvil jaw 158 may begin in the open position given that no activation signal is being sent from the first trigger 140. As will be appreciated, the end effector of the surgical instrument can be understood as a peripheral apparatus or device that may be configured to interact with a task performed by a physician. In some embodiments, the end effector 152 of the surgical instrument 100 of the present disclosure may be configured to interact with a patient's tissue via tissue cutting and surgical stapling of a patient's tissue.

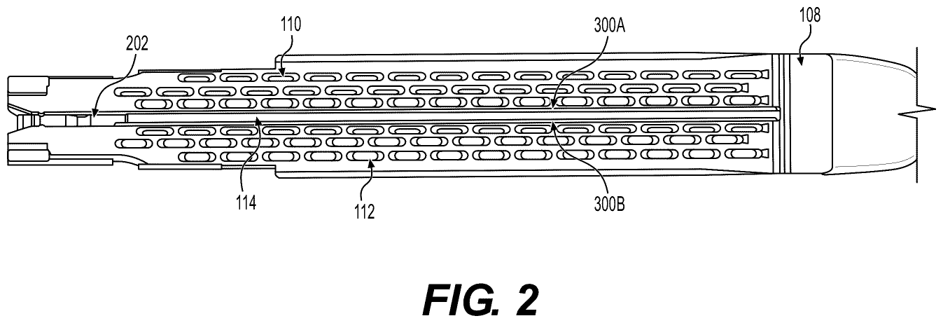

The staple cartridge 200, as indicated by the arrow in FIG. 1, may be disposed within the channel jaw 160 of the end effector 152. In some embodiments, the staple cartridge 200 may include an elongate body 130 and an implantable pouch 300. The elongate body 130 can include a plurality of staples 106, a deck 108, a plurality of staple pockets 110, a central slit 114 within deck 108, and a driver 250. The plurality of staples 106 may be disposed in the plurality of staple pockets 110, wherein each of the plurality of staple pockets 110 may be accessible via an opening 112 defined by the deck 108 of the elongate body 130, as shown in FIG. 2.

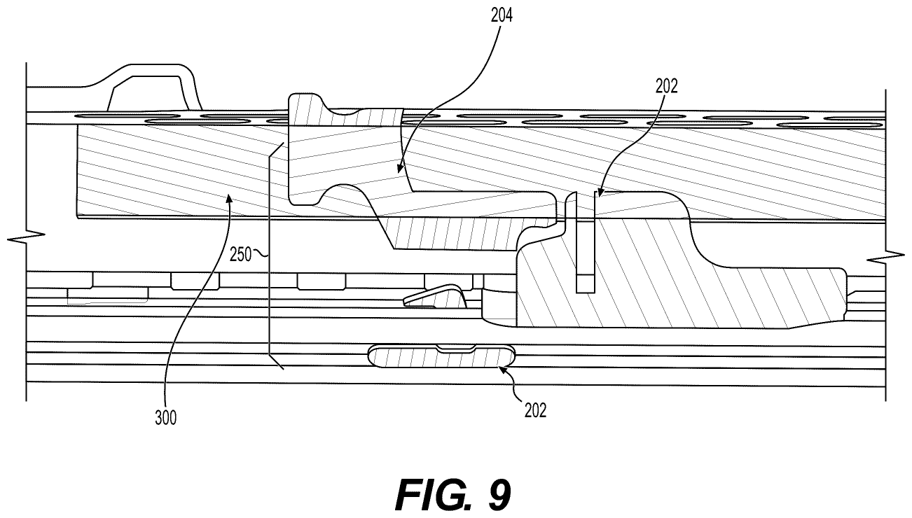

The driver 250 may include at least one of a sled 202 or knife 204 and can be configured to translate through at least a portion of the central slit 114. In some embodiments, the sled 202 and knife 204 can be separate components, wherein the sled 202 can initiate the firing of each of the plurality of staples 106 from the plurality of staple pockets 110 and the knife 204 can traverse the central slit 114 and rupture the implantable pouch 300 to release the medicant 302 onto the cut line of the targeted tissue 170. The embodiment illustrated in FIG. 9, for example, shows the sled and knife as separate components. In other embodiments, the sled 202 and knife 204 can be a single integrated component, wherein the sled/knife act together to initiate the firing of each of the plurality of staples and cutting the implantable pouch 300. Throughout this disclosure, unless otherwise noted, the driver 250 can therefore include one of the sled 202 or the knife 204, or the driver 250 can include both the sled 202 and the knife 204.

In some embodiments, actuating the second trigger 142 can initiate at least one of the sled 202 or knife 204 to translate through at least a portion of the central slit 114. The implantable pouch 300 of the staple cartridge 200 may include a medicant 302, disposed within said implantable pouch 300, and can be removably secured to the elongate body 130. The medicant 302 contained within the implantable pouch may include but not be limited to antibiotics, anti-inflammatory medication, hemostasis agents, anti-bacterial agent, chemotherapy medications, and the like. With respect to chemotherapy medications, classes of medicant 302 may include but not be limited to one or more of alkylating agents, antimetabolites, anthracyclines, corticosteroids, plant alkaloids, and the like.

In some embodiments, the implantable pouch 300 may be positioned on the elongate body 130 to be ruptured by at least one of the plurality of staples 106 or the driver 250. The driver 250 may rupture the implantable pouch 300 and thereby releasing the medicant as it translates through at least the portion of the central slit 114. In some embodiments, the driver 250, may divide the implantable pouch 300 into one or more pieces and thereby release the medicant 302. As illustrated in FIG. 9, the driver 250 can include the knife 204 and the knife 204 cuts the implantable pouch 300 into two pieces. Utilization of an implantable pouch 300 to release medicant 302 may be advantageous as it allows practitioners to provide targeted and controlled delivery of desired deliverable agents in vivo during endoscopic procedures; yet the implantable pouch 300 including the medicant 302 can also remove a second step from the procedure.

Alternatively, the plurality of staples 106 may rupture the implantable pouch 300 as each of the plurality of staples 106 are ejected from each of the plurality of staple pockets 110. In some embodiments, while the end effector 152 is in the closed position, the sled 202 of the driver 250 may move through the central slit 114 when actuated by the second trigger 142, pushing the plurality of staples 106 through the openings 112 in the deck 108 to rupture the implantable pouch 300. Upon rupturing the implantable pouch 300, the medicant 302 may be released on each of the plurality of staples 106.

FIG. 2 is an illustration of a top view of features of a staple cartridge 200. As mentioned previously, the implantable pouch 300 can be positioned on the elongate body 130 to be ruptured by at least one of the driver 250 or the plurality of staples 106. In some embodiments, as shown in FIG. 2, the implantable pouch 300 can be split into one or more pieces 300A, 300B when ruptured by at least one of the driver 250 or the plurality of staples 106. The implantable pouch 300 being ruptured into one or more pieces 300A, 300B can be advantageous as each of the one or more pieces 300A, 300B may include medicant 302 that can chemically mix once ruptured by the driver 250 and plurality of staples 106.

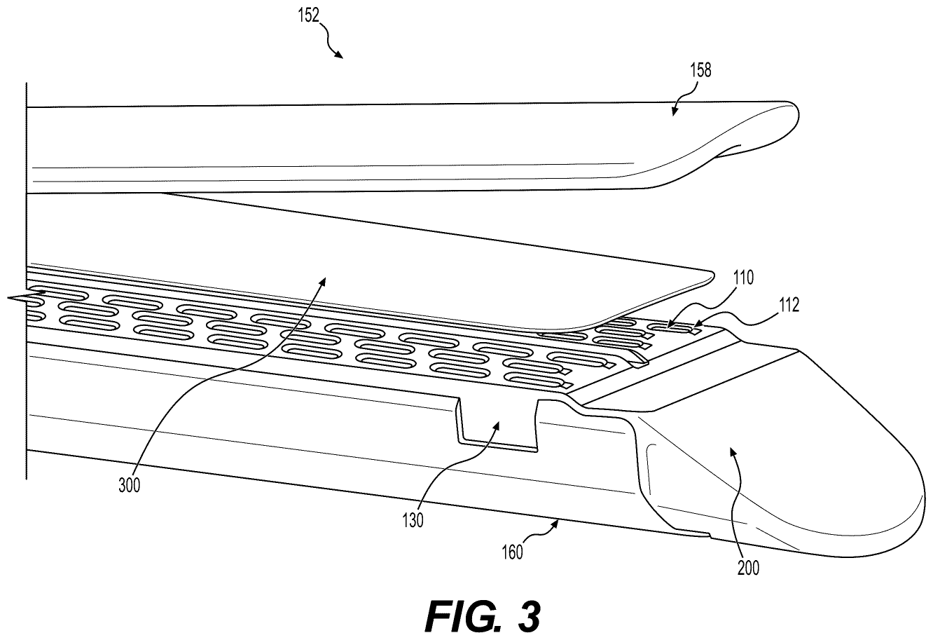

FIG. 3 is a perspective view of the staple cartridge 200 installed within the end effector 152 of a surgical instrument and the implantable pouch 300 while the end effector 152 is in an open position. In some embodiments, as shown in FIG. 3, the implantable pouch 300 may be disposed atop the deck 108 of the elongate body 130 of the staple cartridge 200. Resultantly, the anvil jaw 158 of the end effector 152, upon transitioning to the closed position, may exert a downward, compressive force on the implantable pouch 300 with respect to the channel jaw 160.

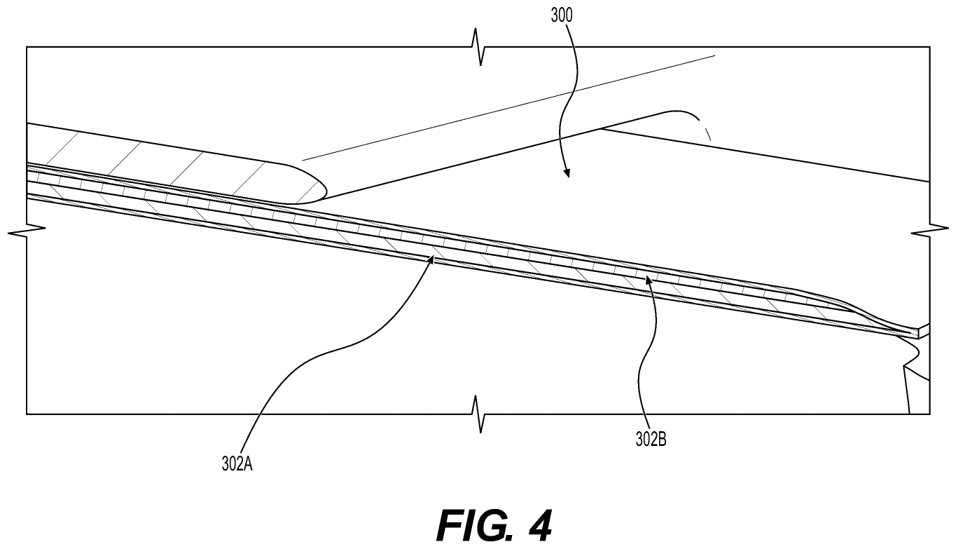

FIG. 4 is a perspective, cross-sectional view of a multi-ply implantable pouch 300 containing a medicant 302 separated into one or more layers 302A,202B prior to being mixed. As one skilled in the art will appreciate, multi-ply pouches contain compartments or one or more layers that are used to house various elements. With respect to the present disclosure, the implantable pouch 300 may be a multi-ply pouch that can house medicant in one or more layers 302A,302B. In some embodiments, once downward pressure is applied by the anvil jaw 158 to the implantable pouch, the one or more layers of implantable pouch 300 can collapse and allow the medicant 302 in the one or more layers 302A,302B to mix. The controlled mixing of the medicant in the one or more layers 302A,302B can be advantageous as it allows the practitioner to control when mixing will occur.

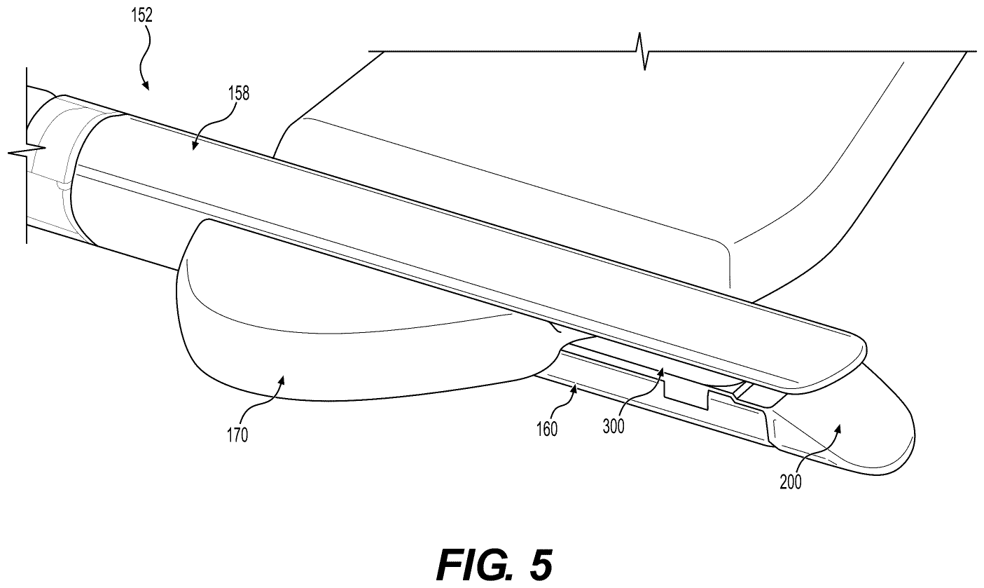

FIG. 5 is a perspective view of a staple cartridge 200 installed within the end effector 152 of the surgical instrument 100 while in the closed position with a targeted tissue 170 disposed therebetween. As one skilled within the art will appreciate, the purpose of clamping targeted tissue 170 with a surgical stapler, as shown in FIG. 5, is to compress the tissue to ensure an even tissue topography prior to cutting and stapling the targeted tissue 170. The risk of not performing appropriate tissue compression may result in poor cutting of tissue and poor hold of surgical staples, which can result in leaks or bleeding after endoscopic procedures. With respect to the present disclosure, the implantable pouch 300 may be a multi-ply pouch that can swell upon medicant 302 mixing within the implantable pouch 300, caused by the application of the downward compressive force by the anvil jaw 158. In some embodiments, the medicant 302 within the implantable pouch 300 may include at least one hydrophilic swellable polymer that can be configured to swell said implantable pouch 300. Swelling the implantable pouch 300 can be advantageous as it promotes increased targeted tissue 170 compression thus decreasing the risk of leakage or bleeding during and after endoscopic procedures.

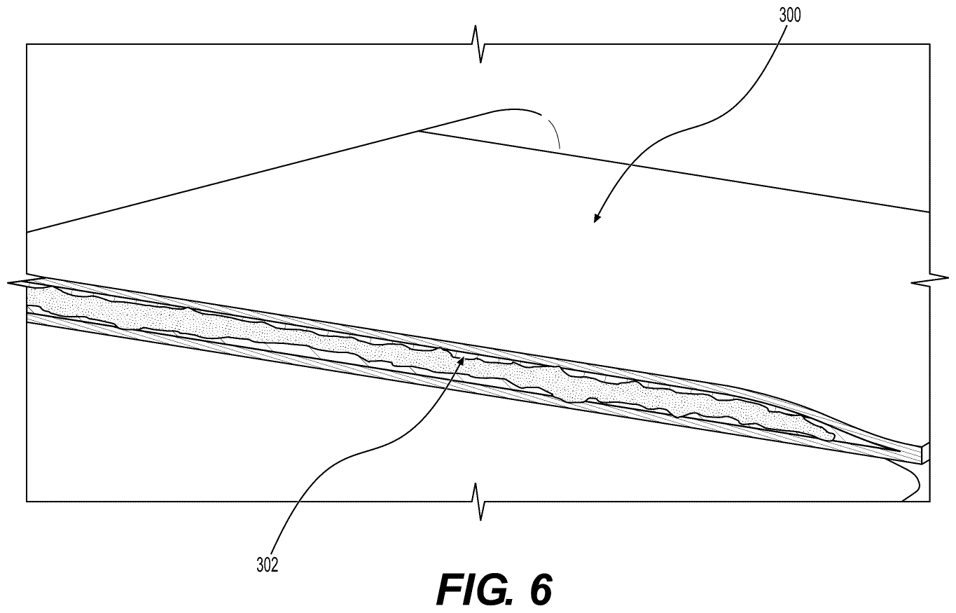

FIG. 6 is a perspective, cross-sectional view of a multi-ply implantable pouch 300 containing medicant 302 in one or more layers 302A,302B after being mixed. As one skilled in the art will appreciate, medicants 302 such as hemostatic agents, coagulant agents, chemotherapy agents, and the like can be multi-part substances that require mixing. Resultantly, through separating the medicant 302 within a multiply implantable pouch 300, practitioners can control mixing in vivo and simultaneously provide targeted, localized treatment upon rupturing the implantable pouch 300 thereby releasing the medicant 302. In some embodiments, practitioners may knead or squeeze the implantable pouch 300 to activate the chemical mixing of the medicant 302 prior to performing endoscopic procedures.

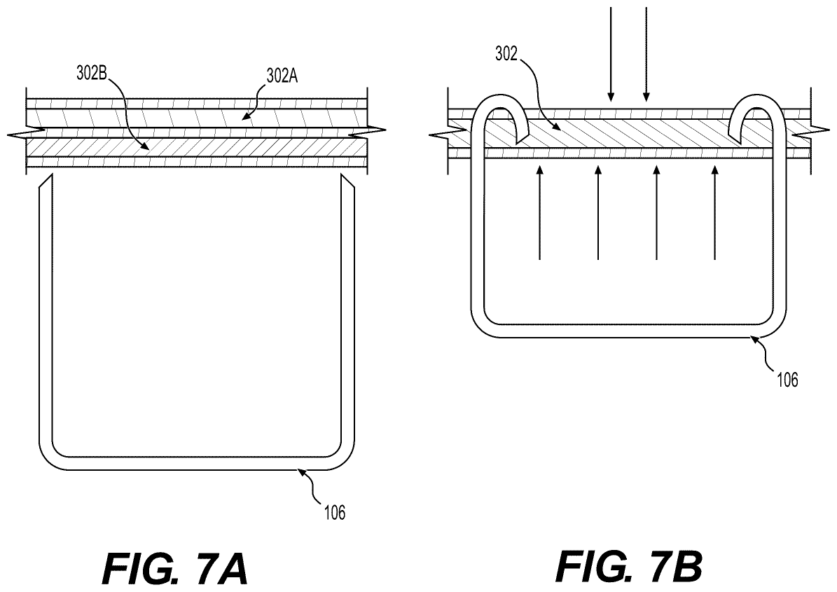

FIGS. 7A-7B are schematics of a multi-ply implantable pouch 300 containing medicant 302 in one or more layers 302A,302B before and after being ruptured by a staple 106, respectively. In some embodiments, as shown in FIG. 7A, the medicant 302 can be suspended within one or more layers 302A,302B within the multi-ply implantable pouch 300 prior to being ruptured by the staple 106. In some embodiments, as shown in FIG. 7B, the staple 106, upon rupturing the implantable pouch 300, can cause the medicant 302 in the one or more layers 302A, 302B to mix and can cause the implantable pouch 300 to swell. Alternatively, rupturing the implantable pouch 300 with the staple 106 can cause the released medicant 302 to react with one or more bodily fluids of the patient. In other words, mixing the medicant 302 of the implantable pouch 300 with blood of the patient can result in swelling and thus promote swelling that can promote hemostasis along the staple line formed by the plurality of staples 106. Examples of medicant that can promote swelling as previously described can include but not be limited to a hydrogel, Metolose®, xanthan, Polyox™, and/or other suitable materials.

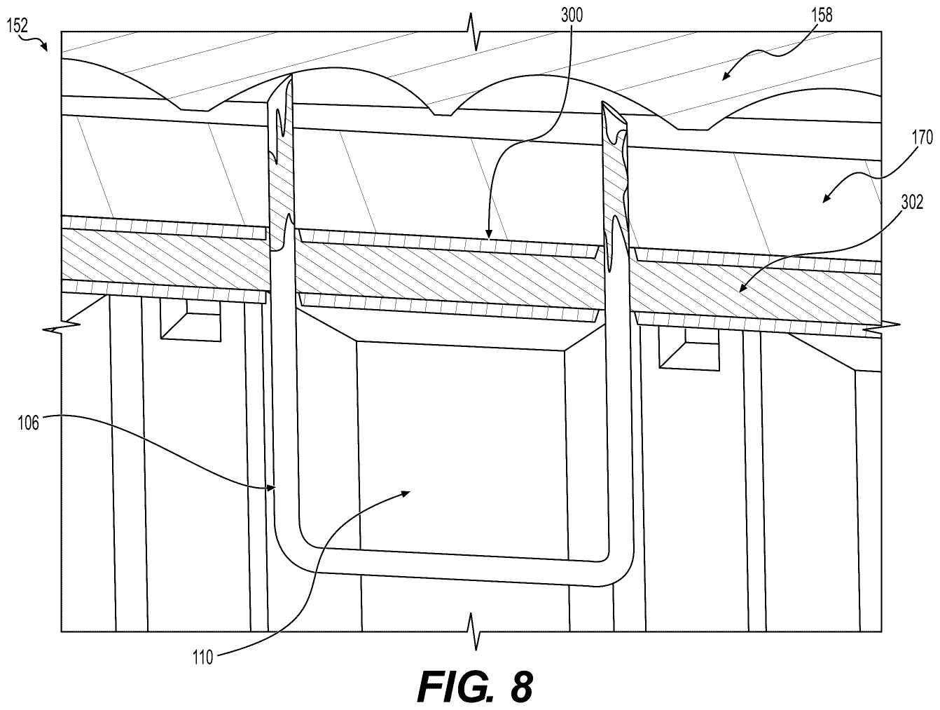

FIG. 8 is an illustration of at least one of a plurality of staples 106 within at least one of the plurality of staple pockets 110 rupturing the implantable pouch 300 containing medicant 302 and carrying the medicant 302 to the targeted tissue 170. As mentioned previously, utilizing the plurality of staple pockets 110 to rupture the implantable pouch 300 and release medicant can be advantageous as it provides targeted delivery of medicant 302 in vivo. As shown in FIG. 8, the medicant 302 coats each of the plurality of staples 106 while moving through the implantable pouch 300 and the targeted tissue 170. Resultantly, once each of the plurality of staples 106 pierces the targeted tissue 170, medicant 302 may be delivered to the targeted tissue 170 while stapling with the surgical stapler.

FIG. 9 is side, cross-sectional view of a loaded staple cartridge 200, showing a driver 250—which in this example includes a sled 202 and a knife 204—configured to traverse central slit 114 of the staple cartridge 200 and rupture an implantable pouch 300 containing a medicant 302. In some embodiments, medicant 302 from the implantable pouch 300 can be applied to targeted tissue 170 during incision by the driver 250 (e.g., sled 202 and/or knife 204). In other words, once the second trigger 142 actuates, either one of the sled 202 and the knife 204 can be initiated and rupture the implantable pouch 300 while traversing the central slit 114. The knife 204, while rupturing the implantable pouch 300, may be coated with the medicant 302. The knife 204, upon reaching the targeted tissue 170, may deliver medicant 302 while cutting said targeted tissue 170. The knife 204 delivering the medicant 302 in conjunction with each of the plurality of staples 106 can be advantageous as it can allow the practitioner to have a greater targeted coverage area for delivering medicant 302 in vivo.

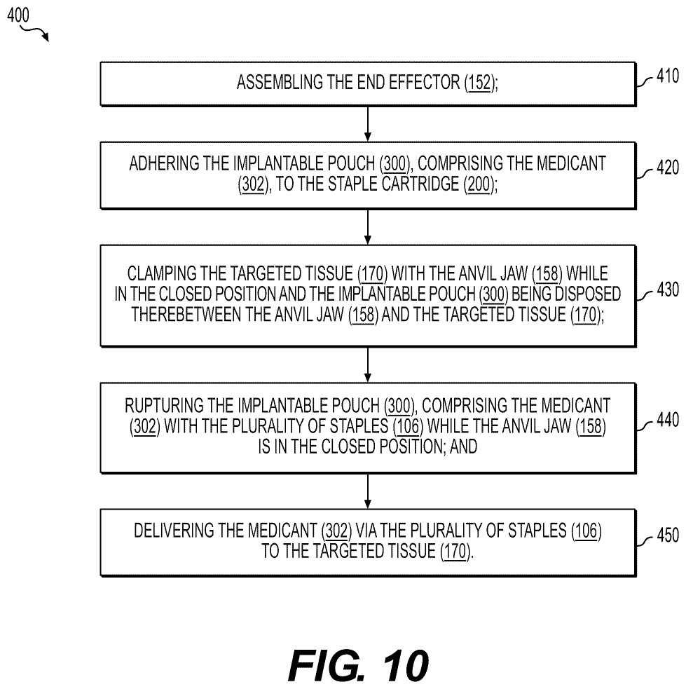

FIG. 10 is a method flow chart for applying medicant 302 onto an end effector 152 of a surgical instrument 100 for delivery to a targeted tissue 170. The method 400 may include the method step 410 of assembling the end effector 152. The end effector 152 may comprise an anvil jaw 158, a channel jaw 160, and a staple cartridge 200 disposed in the channel jaw 160. The anvil jaw 158 may be hingedly connected to the channel jaw 160 and can be moveable between an open position and a closed position with respect to the channel jaw 160. The staple cartridge 200 may comprise an elongate body 130. The elongate body 130 may comprise a plurality of staples 106, a deck 108, a plurality of staple pockets 110, openings 112 defined by the deck, a central slit 114, a driver 250, and an implantable pouch 300.

The driver 250 can comprise a sled 202 and/or a knife 204. The driver 250 can be configured to translate through at least a portion of the central slit 114. Each of the plurality of staple pockets 110 can be accessible via openings 112 of the deck 108. The plurality of staples 106 can be disposed within the plurality of staple pockets 110. The implantable pouch can comprise the medicant 302.

The method 400 can also include the method step 420 of adhering the implantable pouch 300 comprising the medicant 302 to the staple cartridge 200. The method 400 can also include the method step 430 of clamping the targeted tissue 170 with the anvil jaw 158 while in the closed position and the implantable pouch 300 being disposed therebetween the anvil jaw 158 and the targeted tissue 170. The method step 430 of clamping the targeted tissue 170 can also include swelling the medicant 302 within the implantable pouch 300. In some embodiments, the medicant 302 can include at least one hydrophilic swellable polymer that can be configured to swell the implantable pouch 300 when the anvil jaw 158 clamps the targeted tissue 170 with the implantable pouch 300 disposed therebetween.

The method step 430 of clamping the targeted tissue 170 with the anvil jaw 158 can further include mixing the medicant 302 within the implantable pouch 300. In some embodiment, the implantable pouch 300 is a multi-ply pouch and the medicant 302 is separated into one or more layers 302A,302B. The multiply implantable pouch 300 can be configured to mix medicant 302 housed separately in one or more layers 302A, 302B when the anvil jaw 158 clamps the targeted tissue 170 with the implantable pouch 300 disposed therebetween.

The method 400 can include the method step 440 rupturing the implantable pouch 300, comprising the medicant 302 with the plurality of staples 106 while the anvil jaw 158 is in the closed position. The method step 440 of rupturing the implantable pouch 300 can further include pushing the plurality of staples 106 from the plurality of staple pockets 110 through the opening 112 in the deck 108 to rupture the implantable pouch 300. In some embodiments, pushing the plurality of staples 106 from the plurality of staple pockets 110 through the opening 112 in the deck 108 to rupture the implantable pouch 300 can also include coating at least a portion of the plurality of staples 106 with medicant 302. It should be appreciated that the medicant 302 can include but not be limited to at least one of antibiotics, anti-inflammatory medications, hemostasis agents, anti-bacterial agents, chemotherapy medications, and the like. The method 400 can also include the method step 450 of delivering the medicant 302 via the plurality of staples 106 to the targeted tissue 170.

The disclosed technology described herein can be further understood according to the following clauses:

Clause 1: A staple cartridge 200 comprising: an elongate body 130 and an implantable pouch 300. The elongate body 130 comprising: a central slit 114; a driver 250 comprising at least one of a sled 202 or a knife 204, the driver 250 configured to translate through at least a portion of the central slit 114; a deck 108; a plurality of staple pockets 110, each of the staple pockets 110 accessible via an opening 112 defined by the deck 108; and a plurality of staples 106 disposed within the plurality of staple pockets 110. The implantable pouch 300 comprising a medicant 302 disposed therein, the implantable pouch 300 being removably secured to the elongate body 130 and positioned to be ruptured by at least one of: the driver 250 when it translates through at least the portion of the central slit 114, or at least one of the plurality of staples 106 as the at least one of the plurality of staples 106 is ejected from one of the plurality of staple pockets 110; wherein rupturing of the implantable pouch 300 releases the medicant 302.

Clause 2: The staple cartridge 200 according to Clause 1, wherein when the sled 202 moves through the central slit 114 of the staple cartridge 200, the sled 202 pushes the plurality of staples 106 from the plurality of staple pockets 110 through the openings 112 in the deck 108 and thereby rupturing the implantable pouch 300.

Clause 3: The staple cartridge 200 according to any of the preceding Clauses, wherein rupturing the implantable pouch 300 releases the medicant 302 onto the plurality of staples 106.

Clause 4: The staple cartridge 200 according to any of the preceding Clauses, wherein driver 250 ruptures the implantable pouch 300 while moving through the staple cartridge 200.

Clause 5: The staple cartridge 200 according to any of the preceding Clauses, wherein the driver 250 divides the implantable pouch 300 into one or more pieces 300A,300B while moving through the central slit 114 of the staple cartridge 200, releasing the medicant 302.

Clause 6: The staple cartridge 200 according to any of the preceding Clauses, wherein implantable pouch 300 is a multi-ply pouch and the medicant 302 is separated into one or more layers 302A, 302B within the multi-ply pouch.

Clause 7: The staple cartridge 200 according to Clause 6, wherein rupturing the multi-ply implantable pouch 300 mixes the medicant 302 separated into one or more layers 302A, 302B within the multi-ply pouch.

Clause 8: The staple cartridge 200 according to any of the preceding Clauses, wherein the implantable pouch 300 swells responsive to the medicant 302 mixing within the implantable pouch 300.

Clause 9: The staple cartridge 200 according to any of the preceding Clauses, wherein the medicant 302 of the implantable pouch 300 contains at least one hydrophilic swellable polymer.

Clause 10: The staple cartridge 200 according to any of the preceding Clauses, wherein the medicant 302 of the implantable pouch 300 comprises at least one of antibiotics, an anti-inflammatory medication, a hemostasis agent, anti-bacterial agents, or a chemotherapy medication.

Clause 11: The staple cartridge 200 according to Clause 10, wherein the chemotherapy medication is selected from the group consisting of alkylating agents, antimetabolites, anthracyclines, corticosteroids, and plant alkaloids.

Clause 12: In combination, the staple cartridge 200 of any of Clauses 1-11 and an end effector 152 of a surgical instrument, the end effector 152 comprising: a channel jaw 160; an anvil jaw 158 hingedly connected to the channel jaw 160 and movable between an open position and a closed position with respect to the channel jaw 160; wherein the staple cartridge 200 of Clauses 1-11 is disposed in the channel jaw 160.

Clause 13: The end effector 152 of Clause 12, wherein the anvil jaw 158 upon moving to the closed position compresses the implantable pouch 300 thereby causing the medicant 302 to mix within the implantable pouch 300.

Clause 14: The end effector 152 according to Clause 12 or Clause 13, wherein the staple cartridge 200 is configured to releasably attach and detach to at least a portion of the channel jaw 160.

Clause 15: The end effector 152 according to Clause 14, wherein the medicant 302 within each of the one or more implantable pouches 300 is selected from the group consisting of antibiotics, anti-inflammatory medications, and hemostasis agents.

Clause 16: A method 400 for applying a medicant 302 onto an end effector 152 of a surgical instrument 100 for delivery to a targeted tissue 170, the method 400 comprising: assembling the end effector 152, the end effector 152 comprising: a channel jaw 160; an anvil jaw 158 hingedly connected to the channel jaw 160, and is moveable between an open position and a closed position with respect to the channel jaw 160; a staple cartridge 200 disposed in the channel jaw 160 comprising: an elongate body 130 comprising: a central slit 114; a driver 250 comprising at least one of a sled 202 or a knife 204, the driver 250 configured to translate through at least a portion of the central slit 114; a deck 108; a plurality of staple pockets 110, each of the staple pockets 110 accessible via an opening 112 defined by the deck 108; and a plurality of staples 106 disposed within the plurality of staple pockets 110; and an implantable pouch 300 comprising the medicant 302; adhering the implantable pouch 300, comprising the medicant 302, to the staple cartridge 200; clamping the targeted tissue 170 with the anvil jaw 158 while in the closed position and the implantable pouch 300 being disposed therebetween the anvil jaw 158 and the targeted tissue 170; rupturing the implantable pouch 300, comprising the medicant 302 with the plurality of staples 106 while the anvil jaw 158 is in the closed position; and delivering the medicant 302 via the plurality of staples 106 to the targeted tissue 170.

Clause 17: The method 400 according to Clause 16, wherein rupturing the implantable pouch 300, comprising the medicant 302 with the plurality of staples 106 while the anvil jaw 158 is in the closed position comprises: pushing the plurality of staples 106 from the plurality of staple pockets 110 through the opening 112 in the deck 108 to rupture the implantable pouch 300.

Clause 18: The method 400 according to any one of Clauses 16 and Clause 17, wherein pushing the plurality of staples 106 from the plurality of staple pockets 110 through the opening in the deck 108 to rupture the implantable pouch 300 comprises coating at least a portion of the plurality of staples 106 with medicant 302, wherein the medicant 302 comprises at least one of antibiotics, an anti-inflammatory medication, a hemostasis agent, anti-bacterial agents, or a chemotherapy medication.

Clause 19: The method 400 according to any one of Clauses 16 to Clause 18, wherein clamping the targeted tissue 170 with the anvil jaw 158 while in the closed position and the implantable pouch 300 being disposed therebetween the anvil jaw 158 and the targeted tissue 170 comprises swelling the medicant 302 within the implantable pouch 300, wherein the medicant 302 comprises at least one hydrophilic swellable polymer configured to swell the implantable pouch 300 when the anvil jaw 158 clamps the targeted tissue 170 with the implantable pouch 300 disposed therebetween.

Clause 20: The method 400 of any one of Clauses 16 to Clauses 19, wherein clamping the targeted tissue 170 with the anvil jaw 158 while in the closed position and the implantable pouch 300 being disposed therebetween the anvil jaw 158 and the targeted tissue 170 comprises: mixing the medicant 302 within the implantable pouch 300; wherein the implantable pouch 300 is a multi-ply pouch and the medicant 302 is separated into one or more layers 302A,302B; and wherein the multi-ply pouch is configured to mix the medicant 302 separated into one or more layers 302A,302B when the anvil jaw 158 clamps the targeted tissue 170 with the implantable pouch disposed therebetween.

Any of the examples or embodiments described herein may include various other features in addition to or in lieu of those described above. The teachings, expressions, embodiments, examples, etc. described herein should not be viewed in isolation relative to each other. Various suitable ways in which the teachings herein may be combined should be clear to those skilled in the art in view of the teachings herein.

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but in any order as long as the steps allow the embodiments to function for their intended purposes.

Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

1-15. (canceled)

16. A staple cartridge comprising:
an elongate body comprising:
a central slit;
a driver comprising at least one of a sled and a knife, the driver configured to translate through at least a portion of the central slit;
a deck;
a plurality of staple pockets, each of the staple pockets accessible via an opening defined by the deck; and
a plurality of staples disposed within the plurality of staple pockets; and
an implantable pouch comprising a medicant disposed therein, the implantable pouch being removably secured to the elongate body and positioned to be ruptured by at least one of:
the driver when it translates through at least the portion of the central, or
at least one of the plurality of staples as the at least one of the plurality of staples is ejected from one of the plurality of staple pockets,
wherein rupturing of the implantable pouch releases the medicant.

17. The staple cartridge of claim 16, wherein when the sled moves through the central slit of the staple cartridge, the sled pushes the plurality of staples from the plurality of staple pockets through the openings in the deck and thereby rupturing the implantable pouch.

18. The staple cartridge of claim 17, wherein rupturing the implantable pouch releases the medicant onto the plurality of staples.

19. The staple cartridge of claim 16, wherein driver ruptures the implantable pouch while moving through the staple cartridge.

20. The staple cartridge of claim 16, wherein the driver divides the implantable pouch into one or more pieces while moving through the central slit of the staple cartridge, releasing the medicant.

21. The staple cartridge of claim 16, wherein implantable pouch is a multi-ply pouch and the medicant is separated into one or more layers within the multi-ply pouch.

22. The staple cartridge of claim 21, wherein rupturing the multi-ply implantable pouch mixes the medicant separated into one or more layers within the multi-ply pouch.

23. The staple cartridge of claim 16, wherein the implantable pouch swells responsive to the medicant mixing within the implantable pouch.

24. The staple cartridge of claim 16, wherein the medicant of the implantable pouch contains at least one hydrophilic swellable polymer.

25. The staple cartridge of claim 16, wherein the medicant of the implantable pouch comprises at least one of antibiotics, an anti-inflammatory medication, a hemostasis agent, anti-bacterial agents or a chemotherapy medication.

26. The staple cartridge of claim 25, wherein the chemotherapy medication is selected from the group consisting of alkylating agents, antimetabolites, anthracyclines, corticosteroids, and plant alkaloids.

27. In combination, the staple cartridge of claim 16 and an end effector of a surgical instrument, the end effector comprising:
a channel jaw;
an anvil jaw, hingedly connected to the channel jaw, and movable between an open position and a closed position with respect to the channel jaw;
the staple cartridge of claim 16 disposed in the channel jaw.

28. The end effector of claim 27, wherein the anvil upon moving to the closed position compresses the implantable pouch thereby causing the medicant to mix within the implantable pouch.

29. The end effector of claim 27, wherein the staple cartridge is configured to releasably attach and detach to at least a portion of the channel jaw.

30. The end effector of claim 27, wherein the medicant within each of the one or more implantable pouches is selected from the group consisting of antibiotics, anti-inflammatory medications, and hemostasis agents.

31. A method for applying a medicant onto an end effector of a surgical instrument for delivery to a targeted tissue, the method comprising:
assembling the end effector, the end effector comprising:
a channel jaw;
an anvil jaw, hingedly connected to the channel jaw, and is movable between an open position and a closed position with respect to the channel jaw;
a staple cartridge disposed in the channel jaw, comprising:
an elongate body comprising:
a central slit;
a driver comprising at least one of a sled and a knife, the driver configured to translate through at least a portion of the central slit;
a deck;
a plurality of staple pockets, each of the staple pockets accessible via an opening defined by the deck; and
a plurality of staples disposed within the plurality of staple pockets; and
an implantable pouch comprising the medicant;
adhering the implantable pouch, comprising the medicant, to the staple cartridge;
clamping the targeted tissue with the anvil jaw while in the closed position and the implantable pouch being disposed therebetween the anvil jaw and the targeted tissue;
rupturing the implantable pouch, comprising the medicant with the plurality of staples while the anvil jaw is in the closed position; and
delivering the medicant via the plurality of staples to the targeted tissue.

32. The method of claim 31, wherein rupturing the implantable pouch, comprising the medicant with the plurality of staples while the anvil jaw is in the closed position comprises:
pushing the plurality of staples from the plurality of staple pockets through the opening in the deck to rupture the implantable pouch.

33. The method of claim 31, wherein pushing the plurality of staples from the plurality of staple pockets through the opening in the deck to rupture the implantable pouch comprises coating at least a portion of the plurality of staples with medicant,
wherein the medicant comprises at least one of antibiotics, an anti-inflammatory medication, a hemostasis agent, anti-bacterial agents or a chemotherapy medication.

34. The method of claim 31, wherein clamping the targeted tissue with the anvil jaw while in the closed position and the implantable pouch being disposed therebetween the anvil jaw and the targeted tissue comprises swelling the medicant within the implantable pouch,
wherein the medicant comprises at least one hydrophilic swellable polymer configured to swell the implantable pouch when the anvil jaw clamps the targeted tissue with the implantable pouch disposed therebetween.

35. The method of claim 31, wherein clamping the targeted tissue with the anvil jaw while in the closed position and the implantable pouch being disposed therebetween the anvil jaw and the targeted tissue comprises:
mixing the medicant within the implantable pouch; and
wherein the implantable pouch is a multi-ply pouch and the medicant is separated into one or more layers,
wherein the multi-ply pouch is configured to mix the medicant separated into one or more layers when the anvil jaw clamps the targeted tissue with the implantable pouch disposed therebetween.
